(19) United States
(12) Patent Application Publication
(10) Pub. No.: US 2013/0058871 A1
(43) Pub. Date: Mar. 7, 2013

(54) METHOD AND SYSTEM FOR MAPPING SYNAPTIC CONNECTIVITY USING LIGHT MICROSCOPY

(71) Applicant: Jinhyun Kim, Seoul (KR)

(72) Inventor: Jinhyun Kim, Seoul (KR)

(73) Assignee: Howard Hughes Medical Institute, Chevy Chase, MD (US)

(21) Appl. No.: 13/561,882

(22) Filed: Jul. 30, 2012

Related U.S. Application Data
(60) Provisional application No. 61/512,646, filed on Jul. 28, 2011.

(51) Int. Cl. A61K 38/16; C12Q 1/02; C12N 15/63; A61K 31/7088; C07K 14/00; C07H 21/04; A61K 48/00; A61P 35/00; A61P 31/00; C07K 14/435; A61P 25/00; A61P 25/28; A61P 25/16; A61P 25/30; A61P 25/24; G06G 7/48; A61K 49/00

(52) U.S. Cl. 424/9.6; 435/29; 435/320.1; 514/21.2; 514/44 R; 530/350; 536/23.1; 703/11

(57) ABSTRACT

A mammalian Green Fluorescent Protein (GFP) Reconstruction Across Synaptic Partners (mGRASP) method and system is provided by optimizing synaptic transmelectron microscopicbrane carriers for mammalian synapses. The method and system in one form integrates molecular and cellular approaches with a novel computational strategy to reliably reconstruct neurons in 3D. The method and system allows mGRASP to be applied to both long-range and local microcircuits, analysis of synaptic distribution at the level of single neurons and dendritic compartments within neural cells.

### CROSS REFERENCE TO RELATED APPLICATIONS

This application claims benefit of U.S. Provisional Application No. 61/512,646, filed Jul. 28, 2011, the entire disclosure of which is incorporated herein by this reference.

### FIELD OF THE INVENTION

The present invention relates to the method and system for mapping synaptic connectivity and in particular a method and system for mapping synaptic connectivity using light microscopy and pre- and post-synaptic protein molecules.

### BACKGROUND OF THE INVENTION

Over the past century, the desire to link neuronal network activity and behavior has driven neuroscientists to develop increasingly advanced techniques to map the synaptic connectivity within neuronal circuits. Historically the degree of overlap between the axonal arbor of a presynaptic neuron and the dendritic arbor of a postsynaptic neuron has been used to infer the presence of synaptic connectivity. This is a relatively simple determination that follows from the fact that synapses require a physical contact. But because less than half of the axons within reach of a given postsynaptic dendrite actually form synapses, this method is inaccurate and actually only provides a connection probability.

To date, accurately determining the presence and statistical characteristics of an actual synaptic connection requires neuronal reconstruction at the electron microscopic (EM) level. Unfortunately, even with new advances in EM related methodology it remains a relatively time-consuming and volume-limited endeavor to reconstruct a significant region of neuronal tissue. Recently, the development of sophisticated genetic and optical methods, such as array tomography, brainbow and trans-synaptic tracing techniques, have added some additional but still limited capabilities.

Another recent genetically related method to resolve synapses at the level of light microscopy (LM), termed GRASP (Green Fluorescent Protein (GFP) Reconstruction Across Synaptic Partners), is based on two non-fluorescent split-GFP fragments (called sp1-10 and sp11) tethered to the membrane in two neuronal populations. When two neurons, each expressing one of the fragments, are tightly opposed through a synaptic cleft, fluorescent GFP is reconstituted. Thus, using this technique, fluorescence indicates quickly, definitively, and with high spatial resolution, the locations of synapses. Although the GRASP technique has been shown to work well in C. elegans and Drosophila, before GRASP can be used as a transmembrane proximity detector for visualizing synapses in the mammalian brain, a number of significant modifications are required because synaptic architecture varies greatly across organisms.

### SUMMARY OF THE INVENTION

The present invention relates to a method and system for using mGRASP to map synaptic connectivity using light microscopy. The method and system includes using novel synaptic transmembrane carriers for mammalian synapses. The transmembrane elements include a pre-synaptic protein and a post-synaptic protein in which one or both are labeled with a fluorescent marker. The protein molecules can be introduced to neuronal tissue to observe synapsis using light microscopy.

The present method and system can be used with computer-based three dimensions (3D) reconstruction techniques to construct a 3D image of neurons, e.g., generate a 3D brain atlas.

Further, the present method of synapses visualization and three dimensional reconstruction allows one to observe anomalies which one can then be treated using an appropriate therapy.

The present invention, in one form thereof, relates to a protein molecule comprising a signal protein, an extracellular domain, a transmembrane domain and an intracellular domain, from N-terminus. The signal peptide comprises amino acid residues of N-terminus of the nematode β integrin, the extracellular domain and the transmembrane domain comprise consecutive 218 to 250 amino acids within CD4-2 comprising the amino acids from 25^{th} to 242^{nd} positions and the intracellular domain comprises consecutive 55 to 80 amino acids within neurexin 1β comprising the amino acids from 414^{th} to 468^{th} positions.

The present invention in another form thereof relates to a method of pre-synaptic delivery of a bioactive material comprising administering a protein molecule comprising a signal protein, an extracellular domain, a transmembrane domain and an intracellular domain, from N-terminus. The signal peptide comprises amino acid residues of N-terminus of the nematode β integrin, the extracellular domain and the transmembrane domain comprise consecutive 218 to 250 amino acids within CD4-2 comprising the amino acids from 25^{th} to 242^{nd} positions and the intracellular domain comprises consecutive 55 to 80 amino acids within neurexin 1β comprising the amino acids from 414^{th} to 468^{th} positions. Alternatively, the method may include administering an expression vector which expresses a polynucleotide encoding the protein molecule.

The present invention, in another form thereof, relates to a protein molecule comprising a signal peptide, an extracellular domain, a transmembrane domain, and an intracellular domain, from N-terminus. The signal peptide comprises consecutive 49 to 60 amino acid residues within neuroligin comprising the amino acids 1^{st} to 49^{th} position, the extracellular domain comprises consecutive 71 to 85 amino acids within neuroligin comprising amino acids from 627^{th} to 697^{th} positions, the transmembrane domain comprising consecutive 19 to 30 amino acids within neuroligin comprising the amino acids from 698^{th} to 716^{th} positions and the intracellular domain comprises consecutive 127 to 140 amino acids of C-terminus of neuroligin.

The present invention, in another form thereof, relates to a method of preventing or treating a cranial nerve disorder comprising administering at least one of the pre-synaptic a post-synaptic protein molecules described above, i.e., one having a signal polypeptide, an extracellular domain, a transmembrane domain and an intracellular domain comprising nematode and neurexin or the polypeptide comprising neuroligin and a gene or drug for preventing or treating the cranial nerve disorder, to a patient in need of prevention or treating a cranial nerve disorder. By being able to visualize synapses, one can observe anomalies which one can then address using an appropriate therapy.

The present invention, in another form thereof, relates to a method for pre-synaptic delivery of a bioactive material comprising administering the aforementioned pre-synaptic protein molecule comprising nematode, Neurexin 1β, and CD4-2 or an expression vector expressing polypeptide encoding the aforementioned protein, together with a bioactive material, to a patient in need of the administration of the bioactive material.

The present invention, in another form thereof relates to a method of post-synaptic delivery of a bioactive material comprising administering one of the post-synaptic protein molecules described above comprising neuroligin or an expression vector expressing a polypeptide encoding the protein, together with a bioactive material, to a patient in need of administration of the bioactive material.

In alternative forms, the cranial nerve disorder is selected from the group consisting of Alzheimer's disease, autism spectrum disorder, Parkinson's disease, addiction, depression, amyotrophic lateral sclerosis (ALS), and attention deficit hyperactivity disorder.

The present invention, in another form thereof relates to a method for visualizing neurons comprising administering the pre- or post-synaptic protein molecules described above (i.e., a protein molecule comprising nematode, CD4-2 and Neurexin or the protein comprising neuroligin), or an expression vector expressing a polynucleotide encoding the aforementioned proteins, to a subject, wherein the protein is labeled with a fluorescent material or the expression vector comprises a gene for a fluorescent protein at the C-terminus of a signal peptide.

In one further form, the method is adapted for pre-synaptic visualization which comprises administering the protein molecule comprising nematode, CD4-2 and Neurexin or an expression vector expressing a polypeptide encoding the aforementioned protein molecule, to a subject, wherein the protein is labeled with a fluorescent material or the expression vector comprises a gene for a fluorescent protein between a signal polypeptide and an extracellular domain or a linker. In an alternative further form, the method is adapted for post-synaptic visualization and the method comprises administering the post-synaptic protein molecule comprising neuroligin or an expression vector expressing a polypeptide encoding the aforementioned post-synaptic protein, to a patient wherein the protein is labeled with a fluorescent material or the expression vector comprises a gene with a fluorescent protein between a signal polypeptide and an extracellular domain.

The present invention, in another form thereof, relates to a composition for visualizing a neuron comprising at least one of the aforementioned pre-synaptic or post-synaptic protein molecules or an expression vector expressing a polynucleotide encoding the protein(s), wherein the protein(s) is/are labeled with a fluorescent material or the expression vector comprises a gene for a fluorescent protein at a C-terminus of a signal peptide.

The composition in one further form can be adapted for pre-synaptic visualization in which the protein molecule or an expression vector expressing a polynucleotide encoding the protein is labeled with a fluorescent material or the expression vector comprises a gene for a fluorescent protein between a signal polypeptide and an extracellular domain or a linker. In an alternative further embodiment, the composition is for post-synaptic visualization and contains the protein molecule comprising neuroligin or an expression vector expressing a polynucleotide encoding the protein comprising neuroligin, wherein the protein is labeled with a fluorescent material or the expression vector comprises a gene for a fluorescent protein between a signal peptide and an extracellular domain.

The present invention in another form thereof relates to a protein molecule comprising nematode, CD4-2 and neurexin.

The present invention, in another form thereof relates to a post-synaptic protein molecule as described above comprising neuroligin or an expression vector expressing a polynucleotide encoding the protein for use in post-synaptic visualization wherein the protein is labeled with a fluorescent material or the expression vector comprises a gene for a fluorescent protein between a signal peptide and an extracellular domain.

### BRIEF DESCRIPTION OF THE DRAWINGS

FIG. 1 comprises a plurality of panels depicting synaptic mGRASP components and gene delivery strategy wherein, panel (a) is a schematic illustration of mGRASP in the synapse, panel (b) is a diagram of pre- and post-mGRASP composed of signal peptide (SP), split-GFP fragment (spGFP1-10 or spGFP11), extracellular domain, transmembrane domain (TM), and intracellular domain followed by fluorescent proteins (mCerulean, 2A-mCherry, and 2A-dTomato) to allow visualization (scale bar: 50 amino acids (aa)). 2A; 2A-peptide sequence; nls: nuclear localization sequence, panel (c) depicts a strategy for cell-type specific and sparse gene delivery, and panel (d) depicts an example showing both sparse and dense delivery of mGRASP components in the hippocampal CA3-CA1 connection in which merged image shows dense axonal projections of CA3 infected with pre-mGRASP (aavCAG-pre-mGRASP-mCerulean) (blue) and sparse CA1 pyramidal neurons expressing post-mGRASP (aavCAG-Jx-rev-post-mGRASP-2A-dTomato) (red).

FIG. 2 comprises a plurality of panels depicting synaptic expression of mGRASP components wherein panel (a) depicts distribution of pre-mGRASP (aavCAG-pre-mGRASP-mCerulean-2A-nls mCherry) visualized with blue fluorescent signals from mCerulean. Overview of the hippocampus (left) and high magnification images of sub-regions (CA3a,b,c, CA1, and DG, right) show infected cell nuclei in red and their axonal projections in blue. Clear primary axonal expression of pre-mGRASP is visible, while little or no dendritic structure can be seen in blue. ori: stratum oriens; pyr: stratum pyramidale; rad: stratum radiatum; hil: hilus; gr: stratum granulosum; ml: stratum moleculare, panel (b) depicts the highly restricted dendritic distribution of post-mGRASP (paavCAG-post-mGRASP-2A-dTomato) in CA1 pyramidal neurons visualized by fluorescent immunostaining using polyclonal anti-GFP. Little or no immunosignal from post-mGRASP in alveus is evident compared to the strong cytosolic signal from dTomato (indicated by an arrow, left). High magnification images show that post-mGRASP appears to be enriched in postsynaptic density (PSD) in middle and right panels, and panel (c) depicts) immunolabel EM images verify that pre-mGRASP is highly expressed in axonal termini and post-mGRASP is enriched in PSD, as observed in LM. Asterisks indicate PSD and blue arrows indicate immuno-silver-gold particles.

FIG. 3 comprises a plurality of panels depicting reconstitution of mGRASP in hippocampal CA3-CA1 connectivity wherein panel (a) depicts an overview of the hippocampus shows sparse post-mGRASP (aavCAG-Jx-rev-post-mGRASP-2A-dTomato) labeling of CA1 (red) and dense contralateral pre-mGRASP (aavCAG-pre-mGRASP-mCerulean) projections of CA3 (blue) (left). Discrete puncta of reconstituted mGRASP fluorescence in one CA1 neuron in (yellow dashed box, left) are visible along dTomato-labeled apical and basal dendrites in locations where blue CA3 axons and red CA1 dendrites intersect, but not in tuft dendrites in the striatum lacunosum-moleculare (Im) (right), panel (b) depicts a high magnification images of subclasses of example dendrites from the CA1 neuron in (white dashed box, right, a) show strong mGRASP signals in the spine heads of both distal and proximal basal (1 and 2) and apical oblique (3) dendrites where blue axons intersected with red dendrites, yet there is no signal in tuft dendrites (4), and panel (c) depicts a cropped high magnification images of a proximal basal dendrite distinctly show strong reconstituted mGRASP signals in the spine heads where blue axons intersected with red dendrites (arrow heads).

FIG. 4 comprises a plurality of panels depicting reconstruction of neurons and detection of mGRASP in 3D wherein panel (a) depicts a schematic illustration of the pipeline for mGRASP analysis. Step 1: Tiled high magnification image stacks encompassing entire CA1 neurons were stitched together by computing the maximum correlation coefficient of pixel values in 5-10% overlapping areas. The stitched image was XY-down-sampled by a factor of 2 and color-separated for reconstructing neurons to save processing time. Step 2: A start point (seed) was selected by a mouse click to place the first cylinder (green) using the red channel from step 1. Cylinders (red) were then automatically added one by one along their central axis in both directions. Once cylinders covered the neurite's image by iteration, tubes were connected. Step 3: The radius of each reconstructed neurite cylinder was expanded to the length of spines (on average 2-2.5 μm) and green fluorescent mGRASP puncta was automatically detected within that volume using the full-resolution stitched image from step 1 and the reconstructed neuron from step 2, and panel (b) depicts an example of reconstructed dTomato-labeled CA1 neurons; locations of synapses were automatically detected in 3D through step 1-3 (left). Cropped image illustrating synapse detection (colored spheres) in dendritic branches (right).

FIG. 5 comprises a plurality of panels depicting expression and reconstitution of mGRASP inducing no changes in synaptic organization wherein panel (a) depicts cytosolic dTomato-expressing pre- and post-mGRASP were massively introduced into CA3 of the left hemisphere and CA1 of right, respectively, and reconstituted mGRASP fluorescent signals were detected in both oriens and radiatum of CA1, panel (b) depicts quantitative EM analysis showed that the number of excitatory synapses under EM was no different in hippocampi with reconstructed mGRASP signals compared with only post-mGRASP- and control, non-infected hippocampi.

FIG. 6 comprises a plurality of panels depicting mGRASP detecting actual synapses with high specificity wherein panel (a) depicts a non-synaptic pair (CA3-OLM), unconditional pre-mGRASP and Cre-dependent ‘switch-on’ post-mGRASP (upper left) were injected into CA3 and CA1 of the sst-Cre mouse, separately. For a synaptic pair (CA1-OLM), both Cre-dependent ‘switch-off’ pre-mGRASP and Cre-dependent ‘switch-on’ post-mGRASP (upper right) were injected into CA1 of the sst-Cre mouse at different vertical depths. Merged images show blue axonal projections from CA3 and CA1, and reveal clear post-mGRASP expression in OLM cells. Merged image shows a positive synaptic pair, blue axons of a small fraction of granule cells was additionally detected but the usual pattern of exclusive CA1 axonal projections is shown in FIG. 8, and Panel (b) depicts low to high axon density of CA3 and CA1 surrounding post-mGRASP-expressing OLM cells presented in a color-coded reconstruction; insets indicate locations of OLM cells along with axonal projections (left). As expected, mGRASP puncta were detected in CA1-OLM but few or none were evident in CA3-OLM (right). (c) Quantification of mGRASP density per cell and per dendritic surface area in negative and positive synaptic partners shows very high specificity of synapse detection.

FIG. 7 comprises a plurality of panels depicting distribution of excitatory and inhibitory synapses revealed by mGRASP wherein panel (a) illustrates the result of automated reconstruction and mGRASP detection for two nearby neurons (left). Excitatory and inhibitory synapses were automatically distinguished based upon the size and shape of mGRASP signals in somata and dendrites; image details are shown at right and panel (b) depicts dendrogram illustrating mGRASP detected on a CA1 neuron, showing the lengths of dendrites and locations of synapses in dendritic compartments.

FIG. 8 comprises a plurality of panels depicting primary axonal expression of pre-mGRASP. Serial coronal sections containing hippocampi aligned from anterior to posterior. rAAV expression pre-mGRASP (aavCAG-pre-mGRASP-mCerulean) was injected into CA3 regions (a) and CA1 (b) of the right hemisphere. (a) Blue-labeled axonal projections of CA3 are visible in both ipsi- and contralateral sides of hippocampi. Scale bar: 1 mm. (b) Blue-labeled axons of CA1 were detected exclusively in the alveus of anterior and in subiculum of posterior hippocampus, while somata or dendrites of infected CA1 were hardly visible. Scale bar: 1 mm (left) and 500 μm (right).

FIG. 9 comprises a plurality of panels depicting reconstitution of mGRASP in a long-range circuit, thalamocortical connectivity wherein panel (a) strong and dense post-mGRASP expression (aavCAG-Jx-rev-post-mGRASP-2A-dTomato) in the Six3-Cre line is evident in different cell types of layer 4 (L4) as well as other layers (left). Pre-mGRASP expression (aavCAG-pre-mGRASP-mCerulean) resulting in blue fluorescent axonal projections from the VPM can be seen mostly in L4 but also slightly throughout cortical layers (right). Scale bar: 500 μm. (b) Similar to results obtained with mGRASP in the hippocampus, clear and strong reconstituted mGRASP puncta are clearly visible in sites where dTomato-labeled L4 neurons and blue thalamic axons intersect. Scale bar: 20 μm.

### DETAILED DESCRIPTION

The present invention relates to use of a novel mGRASP neuromapping method and system which uses novel protein molecules. Mapping synaptic connectivity is necessary for understanding the functions of interacting populations of neurons. The present invention integrates molecular and cellular proteins with a new computational strategy to reliably reconstruct neurons in 3D. The method and system utilizes one or both novel proteins produced from the domains of transmembrane neuroproteins. Further, the present method of synapses visualization and three dimensional reconstruction allows one to observe anomalies which one can then be treated using an appropriate therapy.

The present invention relates to a new GRASP for mapping long-range circuits as well as microcircuits in the mammalian brain referred to throughout this disclosure as “mGRASP”. In silico technology was used to designed exemplary chimeric synaptic mGRASP components as pre- and post-synaptic proteins which match the approximately 20 nm wide synaptic cleft of mammalian synapses, without modifying synaptogenesis. The synaptic distribution of the designed pre- and post-synaptic mGRASP components were evaluated with electron microscopic techniques, verified that the reconstitution of mGRASP could be detected in the well-studied Schaffer collateral synapses of the hippocampus, and determined that the mGRASP technique led to no significant change in synaptogenesis. It was further verified that mGRASP specifically detects actual synapses, not potential synapses, by examining sites where synapses are known to be absent even though these sites are fully surrounded by non-targeting axons. In parallel, analysis strategies and computational programs to provide reliably reconstruct neurons in 3D, and to investigate synaptic localizations and detailed subcellular distributions.

Designing mGRASP

The present pre- and post-synaptic proteins allow split-GFP reconstitution over the synaptic cleft without causing spurious synapse formation or inappropriate reconstitution at non-synaptic regions. Proteins selected for the present mGRASP molecules are localized at synapses, namely neurexin and neuroligin. These proteins were selected based on their known transmembrane adhesion molecules, which are enriched in pre- and post-synaptic surface, respectively, and which provide trans-synaptic connectivity. The selected molecules were used to create pre- and post-synaptic mGRASPs. The resulting pre- and post-synaptic mGRASP component (i) are targeted to and are maintained in synapses; and (ii) the molecular lengths of the extracellular domains including split-GFP fragments fit appropriately into the synaptic cleft.

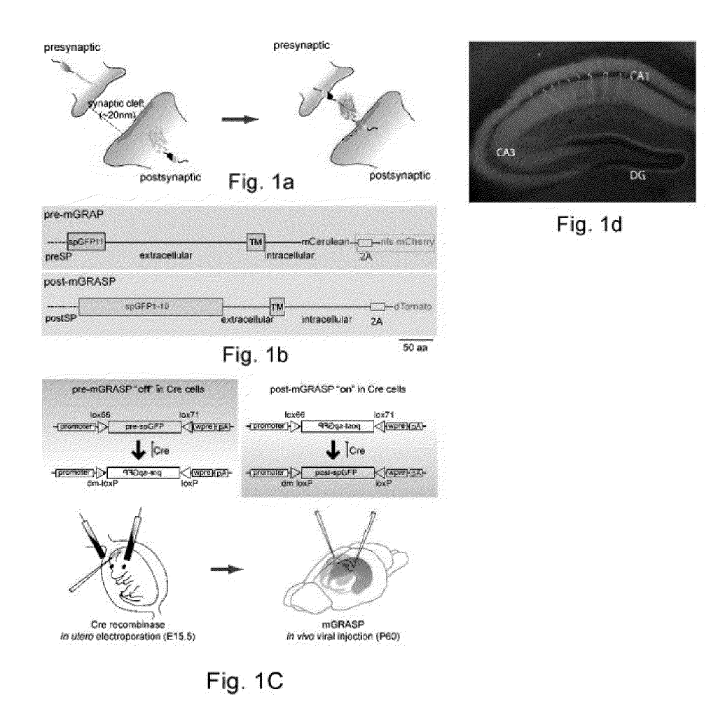

Both pre- and post-synaptic mGRASP components are composed of (1) signal peptide, (2) split-GFP fragment, (3) extracellular domain, (4) transmembrane domain, and (5) intracellular domain followed by fluorescent proteins for visualizing neurites (FIG. 1b). For one exemplary presynaptic mGRASP component (hereafter called pre-mGRASP), the signal peptide was the first 29 residues (1-29) of the nematode β integrin (PAT-3) followed by spGFP11 (16 residues) and two (GGGGS) linkers, the extracellular domain and predicted transmembrane domain of the human CD4-2 (25-242), as in the original GRASP. To target and maintain this construct specifically in presynaptic sites, the protein included an intracellular domain, the 55 residue C-terminus of the rat neurexin 1β (414-468) containing its PDZ-binding motif that is necessary for its exit from ER/Golgi and for synaptic targeting. To visualize the presynaptic component, mCerulean was fused to the construct.

For the postsynaptic mGRASP component (hereafter called post-mGRASP), mouse neuroligin 1 was used as the main skeleton. Neuroligin contains 575 residues of catalytically inactive esterase (52-626) that are known to interact with neurexin, leading to the formation of synapses. Thus, residues 52-626 were deleted to avoid additional synaptic formation via interactions with the endogenous neurexin. The 648 residue spGFP1-10 fragment was inserted after the signal peptide (1-49) of the esterase-truncated neuroligin. Thus, post-mGRASP consisted of the 71 residue extracellular domain, the 19 residue predicted transmembrane domain, and the 127 residue C-terminus of mouse neuroligin. Cytosolic dTomato via the 2A-peptide after post-mGRASP in one construct was expressed to visualize the morphology of the postsynaptic cell.

Strategy of Gene Delivery

Gene delivery required determining how to deliver pre- and post-mGRASP into defined cell populations without co-expressing both mGRASP components in the same cell. To test the pre- and post-mGRASP constructs in an exemplary model, namely the mouse brain, CA3-CA1 connectivity of the hippocampus was used. Additionally, sparse labeling of postsynaptic CA1 neurons was used to enable resolution of individual cells and their dendrites in a way that would be suitable for a subsequent automated reconstruction. To achieve cell-type specific and sparse gene delivery, a combination of Cre recombinase and spatially restricted injection by Cre-dependent or independent viral vectors were used to ipsi- and contralateral sides of the hippocampus (FIG. 1c). First, a faithful Cre-mediated switchable inversion vector was generated by using two mutant loxP sites (lox66 and lox71) oriented in a head-to-head position. When Cre recombinase is present, the gene flanked by the two mutant loxP sites is inverted, forming one loxP and one double-mutated loxP site. Because the double-mutated loxP site shows very low affinity for Cre recombinase, the favorable one-step inversion is nearly irreversible, allowing the gene to be stably switched ‘on’ and ‘off’ as desired (FIG. 1c). Leakiness of expression in the absence of Cre was minimized by eliminating sequences containing false TATA boxes and start codons at the sides of the flexed gene.

The plasmid containing iCre recombinase under the control of the CAG promoter was introduced into hippocampal CA1 progenitor cells on embryonic 15.5 day via in utero electroporation. The success rate of hippocampal CA1 transfection was over 85%, determined by in utero electroporation of the plasmid containing fluorescent proteins (EGFP or tdTomato, data not shown). Approximately 2 months after the in utero electroporation, we then injected AAV-expressing pre-mGRASP (aavCAG-pre-mGRASP-mCerulean) into CA3 neurons, and AAV-expressing Cre-dependent ‘switch on’ post-mGRASP (aavCAG-Jx-rev-post-mGRASP-2A-dTomato) into CA1 neurons. Thus achieving selective and sparse labeling in approximately 50-200 CA1 pyramidal neurons without overlap in CA3 (FIG. 1d). This strategy allowed: (1) control of sparseness of labeling by combining two different ways of delivering genes; (2) avoidance of long-term expression of exogenous synaptic proteins; and (3) a wide choice of cell-specific expression using Cre-dependent virus, allowing access to a number of well-characterized pre-existing and upcoming Cre transgenic mouse lines.

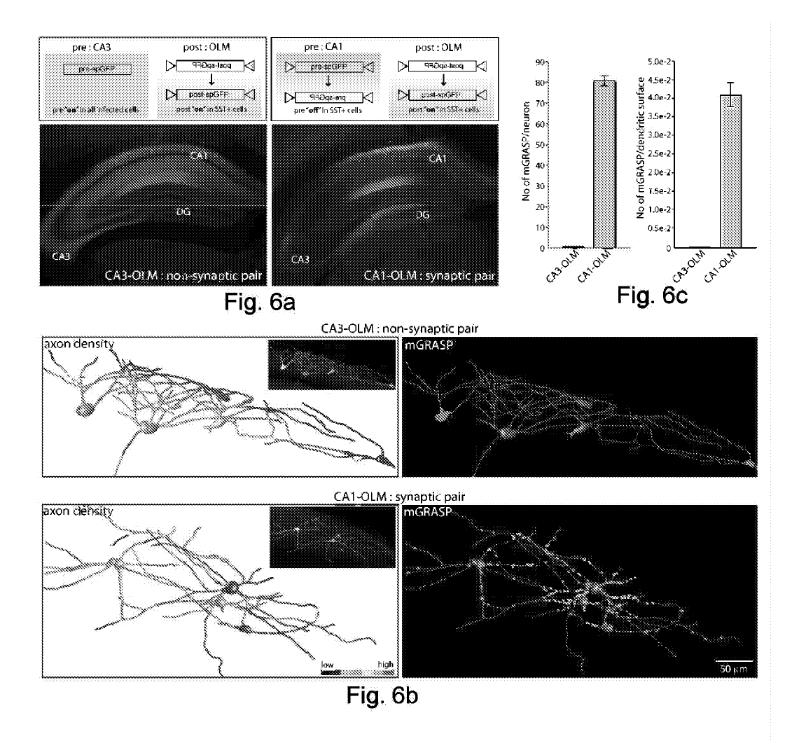

An advantageous labeling technique was developed to label two distinct cell populations that are spatially close to each other. Cre-dependent “switch off” pre-mGRASP, and “switch on” post-mGRASP was applied to cell-type specific Cre mouse lines, as illustrated in FIG. 1c and FIG. 6a. This strategy allows mapping local connectivity.

Synaptic Expression of Designed Pre- and Post-mGRASP

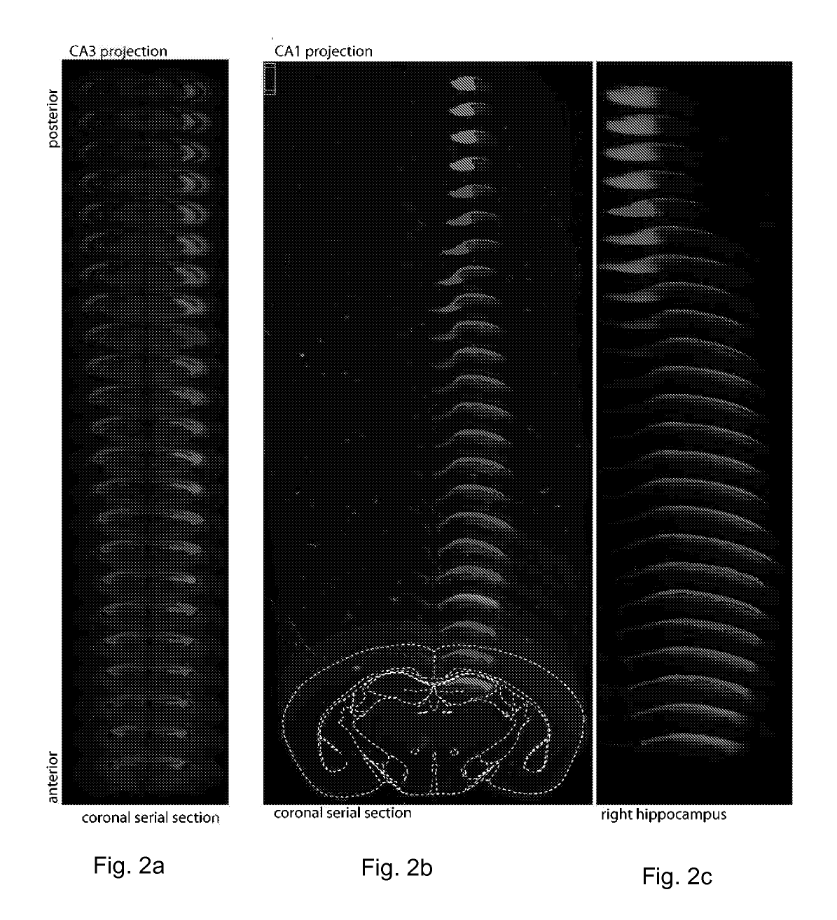

To determine the synaptic expression of pre- and post-mGRASP, these proteins were introduced separately into CA3 and CA1 neurons in the mouse hippocampus, and examined their distribution using LM and EM techniques (FIG. 2). In the CA3 region injected with AAV-expressing pre-mGRASP fused with mCerulean (aavCAG-pre-mGRASP-mCerulean), blue fluorescent signals were only detectable in axonal projections, making it difficult to identify infected neurons. Thus, to facilitate visualizing infected cells we generated a new construct including mCerulean-fused pre-mGRASP followed by the 2A-peptide with nucleus-localized mCherry (aavCAG-pre-mGRASP-mCerulean-2A-mCherry) (FIG. 1b). Under LM, strongly labeled blue axons were visible in both ipsi- and contralateral sides of hippocampi, and high magnification images of infected CA3 and DG areas and non-infected CA1 area clearly showed primary axonal expression of pre-mGRASP (FIG. 2a). In addition, under EM, silver-gold immunolabeling of anti-GFP against mCerulean allowed us to confirm that the pre-mGRASP component was effectively targeted to presynaptic sites (FIG. 2c).

Immunofluorescence staining was used to check post-mGRASP expression. Since post-mGRASP comprises most of the β-barrel structure of GFP (sp1-10), many commercially-available polyclonal antibodies against GFP can recognize post-mGRASP. A plasmid containing post-mGRASP (paavCAG-post-mGRASP-2A-dTomato) was transferred into CA1 cells via in utero electroporation and visualized its expression pattern by immunostaining with anti-GFP (FIG. 2b). The expression of post mGRASP appeared to be highly restricted to dendritic branches and it was not detectable in axons of CA1 neurons. In the alveus, which contains the myelinated axons of CA1 pyramidal neurons, little or no immunosignal from post-mGRASP could be detected compared to the cytosolic signal from dTomato. Further, high-magnification images under LM, and immunolabeled images under EM showed that post-mGRASP is highly enriched in the postsynaptic density (PSD) (FIG. 2b and FIG. 2c). It is also important to note that both pre- and post-mGRASP appeared to be expressed through long neurites as we detected pre-mGRASP along CA3 axonal fibers several millimeters in length, and post-mGRASP up to the ends of both apical and basal dendrites of CA1 pyramidal neurons. Taken together, mGRASP components were successfully targeted into synaptic sites as intended, and their expression patterns indicate mGRASP is appropriate for mapping long-range circuits.

Reconstitution of mGRASP in the Mouse Brain

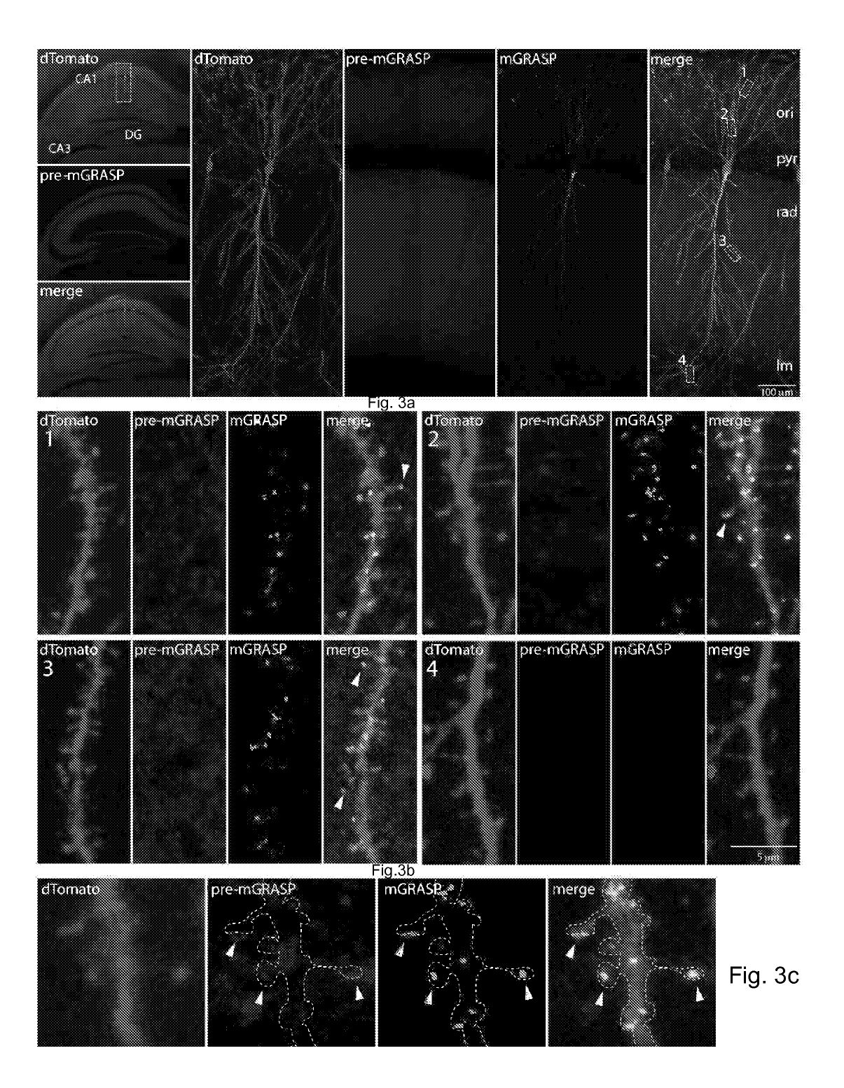

The following study was performed to confirm correct reconstruction in the mouse brain, thus confirming suitability in mammalian brain tissue. To reconstruct postsynaptic neurons, postsynaptic CA1 neurons were labeled sparsely while densely labeling presynaptic CA3 neurons. As describe above, sparse labeling of CA1 was achieved by combining two gene delivery methods and a Cre-dependent gene switch. To completely avoid co-expressing both mGRASP components in the same neuron, contralateral projections of CA3 were used in a first test. In utero electroporation provided plasmid containing iCre recombinase (paavCAG-iCre) to the right ventricle of embryos, and, two months later, injected AAV-expressing pre- (aavCAG-pre-mGRASP-mCerulean) and Cre-dependent post-mGRASP (aavCAG-Jx-rev-post-mGRASP-2A-dTomato) into CA3 of the left hemisphere and CA1 neurons of the right hemisphere in the same animal. While neither split-GFP fragments fluoresced individually, mGRASP was successfully reconstituted trans-synaptically, revealing discrete puncta of fluorescence along dTomato-labeled CA1 apical and basal dendrites in locations where mCerulean-labeled blue CA3 axons and red CA1 dendrites intersect (FIG. 3a). The fluorescence signals of reconstituted mGRASP were clearly evident in both the apical and basal dendritic structures of a CA1 neuron, while no signals were evident along tuft dendrites in the striatum lacunosum-moleculare where axons from CA3 do not project (FIG. 3b). High magnification images showed strong mGRASP signals in the spine heads of both apical and basal dendrites where blue axons intersected with red dendrites (FIG. 3c).

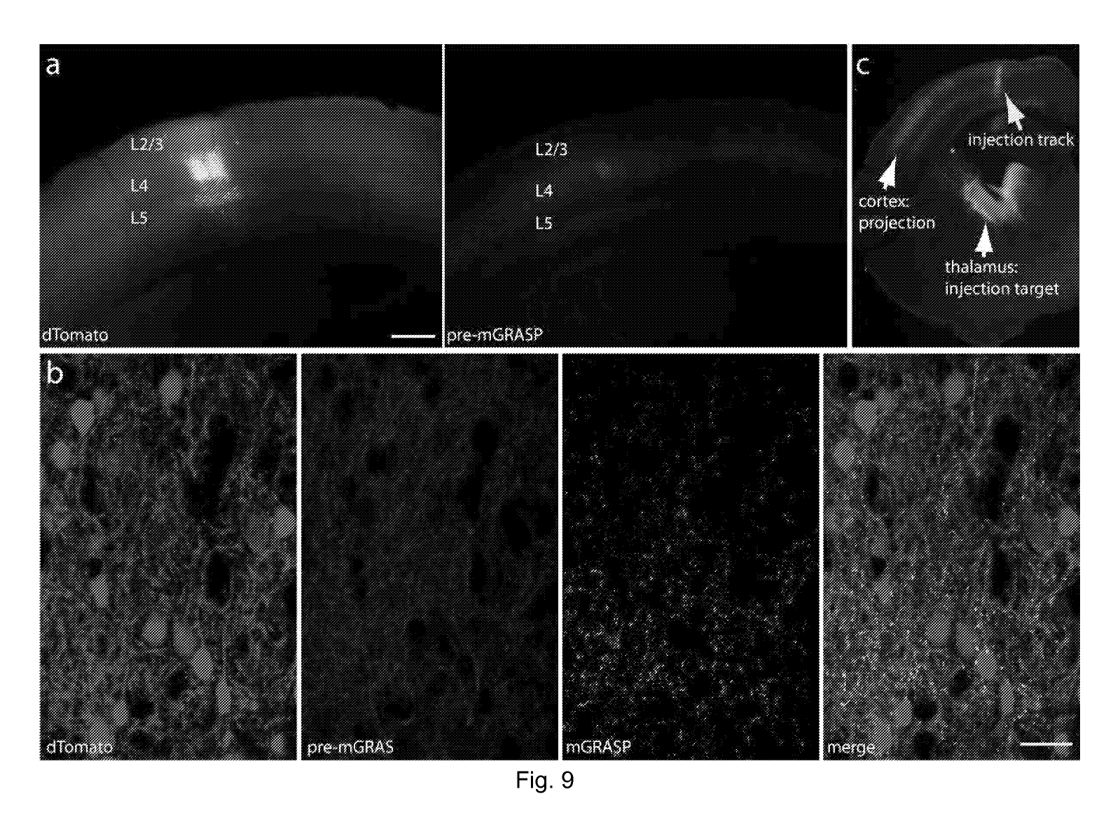

Furthermore, mGRASP reconstitution was tested in another long-range circuit, the thalamocortical circuit (VPM-L4). Pre-mGRASP was introduced into the ventral posterior medial nucleus (VPM) of thalamus and Cre-dependent post-mGRASP into somatosensory cortex of the Six3Cre mouse line expressing Cre recombinase mainly in layer 419. Blue fluorescent axonal projections of pre-mGRASP from the VPM were observed mostly in L4 but also less densely throughout all cortical layers, while strong and dense labeling of post-mGRASP was detected in L4 and other layers (FIG. 9). Similar to results obtained with mGRASP in hippocampus, clear and strong reconstituted mGRASP puncta were detected in sites where dTomato-labeled L4 neurons and blue thalamic axons intersected.

Digital Reconstruction of Neurons and Detection of mGRASP

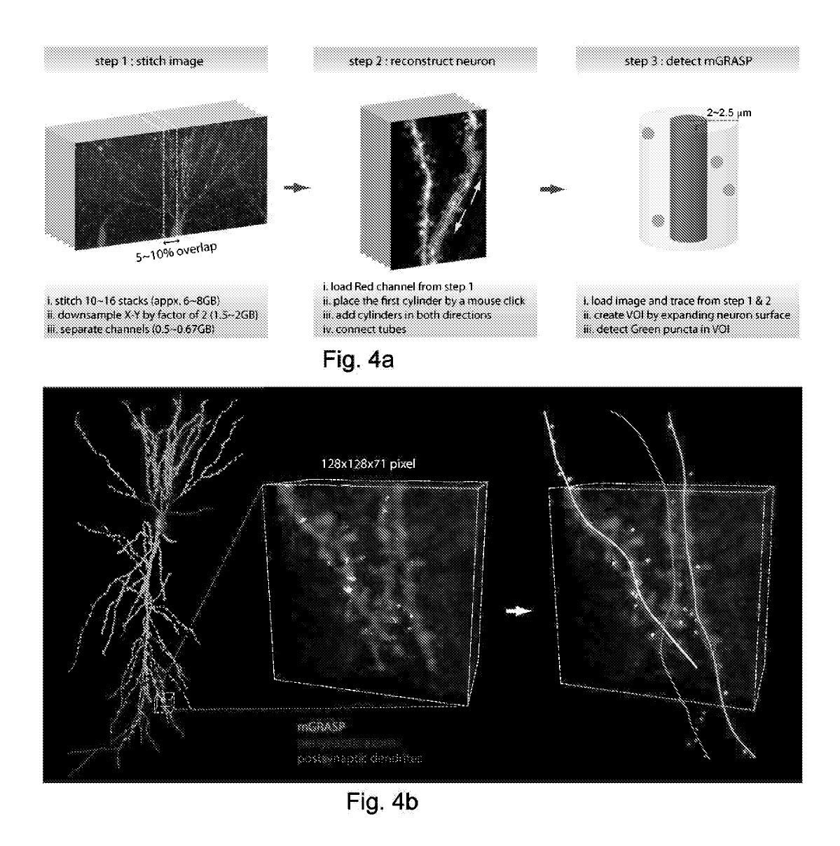

To investigate the localization and distributions of synapses using mGRASP at the level of dendritic compartments as well as single cells, novel analysis strategies and computational programs were developed (FIG. 4a). A pipeline for mGRASP analysis has three main steps: (1) stitch images together; (2) reconstruct neurons; and (3) detect mGRASP puncta. First, because the field of view with high-magnification is too small to encompass an entire CA1 pyramidal neuron, a large field was scanned with multiple tiles by translating the specimen stage. Each image tile consisted of 1024×10²⁴ pixels, covering a field of 106×10⁶ μm with 100-200 vertical steps. The tiles then needed to be properly stitched together to reconstruct entire neurons. Although the microscope system tracks specimen stage movements, the recorded positions were often not sufficiently accurate for high-magnification images. Instead, the positions were estimated from the overlap of image content. Each image tile contained 5-10% overlapping areas from neighboring tiles, allowing their intensity distributions to be used for determining the proper relative positions of each image by computing the maximum correlation coefficient of pixel values.

Second, to accurately reconstruct neuron morphologies, an algorithm optimized for 3D fluorescence images was developed. The algorithm takes advantage of the fact that short orthogonal segments of most neurites approximate elliptical cylinders and neurites resemble extended cylinders forming smooth long tubes. The program, referred to as “neuTube”, initiates the tracing of neuronal morphology from a user selected starting point (seed) using a computer processor. The program, via the processor, places a cylinder on the seed, adjusting its orientations and shape by maximizing the correlation between the cylinder and the image's local intensity distribution. Once the first cylinder is set, it is duplicated and the duplicate is advanced along the cylinder's central axis. The shape and orientation of each cylinder is then refined by a gradient descent algorithm to optimize the match to the underlying neurite image. Cylinders are automatically added one by one along their central axis in both directions until the neurite's image is completely covered. Disjoint neurites are manually indicated by the user. Unlike many other contemporary tracing programs, neuTube does not require explicitly defining branch points. A minimal spanning tree (MST) algorithm is implemented to automatically complete reconstruction by connecting adjacent neurites and by excluding non-tree structures like loops. FIG. 4b shows an example of dTomato-labeled CA1 neurons reconstructed by neuTube from the red channel of a set of stitched images. Reconstructed neurons and detected mGRASP puncta are visualized in vaa3d (http://vaa3d.org/. The connection cost between each pair of the tubes is computed to form a connected graph G of the tubes, in which the weight of an edge is the connection cost between its two nodes. The minima spanning tree (MST) algorithm is applied to the graph to build a tree structure if the tubes. Starting with a new graph T that contains all the tubes and only one edge which is the smallest edge in G, the MST process keeps adding T an edge from G which satisfies: (1) it is not in T; (2) it does not form a loop structure in T; (3) its has smaller weight than any other edge satisfying the first and second conditions. The expansion continues until all the tubes are connected in.

Lastly, a method was developed for automatically detecting mGRASP puncta, allowing us to determine the number and location of synapses along dendrites. First, the radius of each reconstructed neurite cylinder was expanded to the length of spines (on average 2-2.5 μm) since synapses appear within these areas (FIG. 4a). Next, mGRASP fluorescent puncta within this volume of interest were automatically identified. To effectively detect puncta with different sizes, shapes, and densities, a template matching method was used to find best-fit objects.

Preliminary matches are found with respect to a Gaussian kernel modling the smallest possible puncta, and then expanded and shifted with a mean-shift algorithm until the best Gaussian fit covering the initial match is found. This process is carried out with respect to a decreasing sequence of intensity thresholds, where regions matched to puncta at higher thresholds are eliminated from consideration for match at lower levels. Thereafter, a heuristic rule based on the distance and the statistics of the 3D line profile between the centers of two puncta is applied to determine whether two or more neighboring puncta need to be merged. Using our mGRASP detection program, the number and locations of synapses at the level of dendritic branches were automatically detected in 3D with approximately 93.5% accuracy verified by comparison with human annotation of randomly selected sub-volumes (128×128×77 voxels) of neuTube-reconstructed neurons by multiple individuals.

Validation of mGRASP

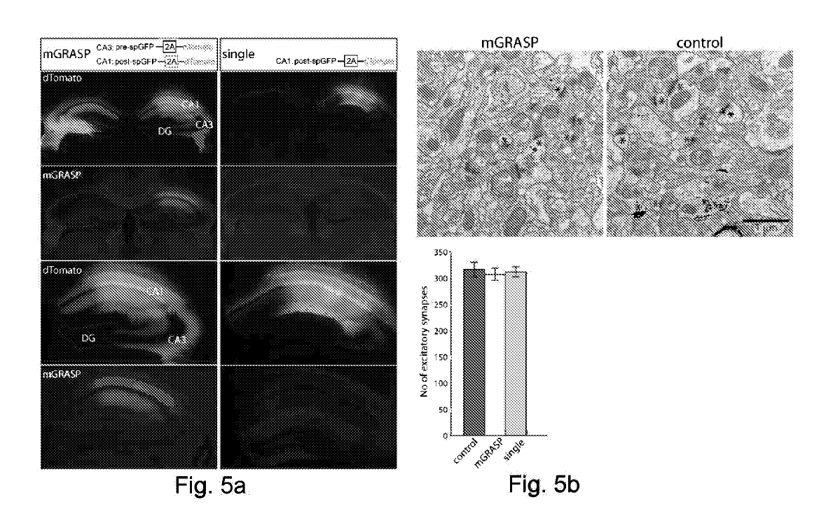

To test whether mGRASP can detect synapses in the mouse brain without introducing artifacts, an investigation was undertaken to determine whether mGRASP induces changes in synaptic organization. Although both pre- and post-mGRASP carriers were designed to avoid interacting with endogenous adhesion molecules, and earlier split-GFP-based reporter system have been shown to induce low or no spontaneous self-affinity, a test was conducted to determine whether mGRASP could promote cell adhesion and cause the artificial formation of additional synapses. For this test, another construct (aavCAG-pre-mGRASP-2A-dTomato) was generated to label pre-mGRASP red, and injected AAV expressing pre- and post-mGRASP (aavCAG-post-mGRASP-2A-dTomato) into CA3 of the left hemisphere, and CA1 neurons of the right hemisphere in the same animal. This resulted in relatively dense labeling of mGRASPs in red, allowing visualization, even under low magnification, reconstituted fluorescent mGRASP signals in what appeared to be most synapses in both oriens and radiatum of CA1 (FIG. 5a). The region of reconstituted mGRASP signals was analyzed with conventional EM to check the synapses (FIG. 5b). Using non-infected hippocampi, and hippocampi infected with only single mGRASP components as controls, no significant differences in the numbers of excitatory synapses (307±22.9, n=5 mice for both mGRASPs, 311.6±19.3, n=5 was found for single mGRASP, and 317±28.23, n=5 for non-infected), and no observable differences in the morphology of CA1 neurons. It is also important to note that our method of gene delivery did not cause any alteration in the number of synapses.

To have a true measure of whether mGRASP detects actual synapses rather than neurite touches, cell populations known to be synaptically connected or not synaptically connected (i.e. CA3 pyramidal neurons-oriens-lacunosum moleculare (OLM) interneurons were analyzed as a non-synaptic pair and CA1 pyramidal neurons—OLM interneurons as a synaptic pair). This test is powerful because axons of both CA3 and CA1 intersect with dendrites of the OLM cells, but mGRASP should detect only actual synaptic contacts from CA1 axonal projections but not from CA3.

To express post-mGRASP selectively in OLM cells, the Cre-dependent “switch-on” post-mGRASP (aavCAG-Jx-rev-post-mGRASP-2A-dTomato) was used in a genetically manipulated mouse line expressing the Cre recombinase under the control of the endogenous somatostatin promoter via knock-in (hereafter called sst-Cre). Labeled OLM cells were identified by their spiny dendrites in the oriens and alveus and their distinctive axonal projections in the striatum lacunosum-moleculare. To label a negative presynaptic partner of the OLM, AAV expressing pre-mGRASP (aavCAG-pre-mGRASP-mCerulean) was injected into CA3. For a positive presynaptic partner of the OLM, the Cre-dependent ‘switch-off’ pre-mGRASP (aavCAG-Jx-pre-mGRASP-mCerulean) was injected into CA1 to avoid co-expressing both mGRASP components in the same cell, as described above, since they are spatially close to one another (see FIG. 1b and FIG. 6a).

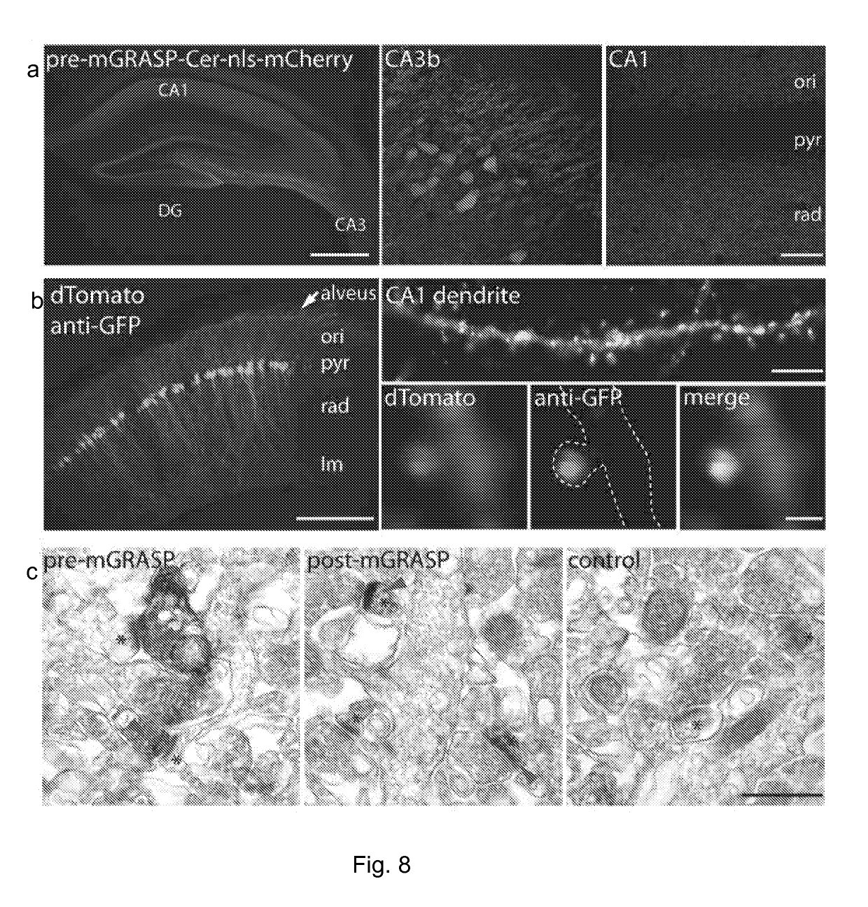

Taking advantage of the exclusive axonal labeling of pre-mGRASP, axonal projections of CA1 pyramidal neurons were identified by their blue fluorescent signals (FIG. 8). neuTube was used to trace post-mGRASP-expressing OLM dendrites, detected mGRASP signals, and then compared detected mGRASP density along the dendrites intersecting with CA1 and CA3 axons (FIG. 6b). The availability of axons in the local environment surrounding OLM dendrites was quantified by measuring the average intensity of blue signal in the same expanded tubes of reconstructed OLM dendrites that we used for mGRASP detection (radius of the traced tube plus ˜2.5 μm).

In the case of the negative synaptic partners CA3-OLM, little or no reconstituted mGRASP puncta was detected, although many axon-dendrite intersects could be seen. Of the few mGRASP puncta detected in CA3-OLM connections, over 78% occurred on somata and likely reflect innervations from other interneurons in CA1. By contrast, many reconstituted mGRASP puncta in CA1-OLM were observed, especially on dendrites of OLM cells. Interestingly, axon density was not well-correlated with mGRASP density. Overall, strikingly clear results in mGRASP detection were found from negative and positive synaptic partners with the same postsynaptic populations, i.e. OLM (mGRASP density per cell; 0.989±0.169, n=61 cells from 3 mice for CA3-OLM and 80.383±4.992, n=65 cells from 3 mice for CA1-OLM: mGRASP density per dendritic surface area (μm2); 1.93E-04±0.28E-04, n=5 stitched image stacks from 3 mice for CA3-OLM and 4.41 E-02±0.66E-04, n=5 stitched image stacks from 3 mice for CA1-OLM) (FIG. 6c). Thus, as predicted, mGRASP signals were detected exclusively in CA1-OLM not in CA3-OLM connections. This provided additional evidence excluding the possibility that mGRASP could cause the artificial formation of synapses. Together, these results clearly indicate that with high specificity, mGRASP detects actual synapses rather than neurite touches, and induces no artifactual effects on synaptic organization. Thus mGRASP expression satisfactorily fulfills our criteria of specific labeling actual synapses without inducing aberrant synapse formation.

Determining Distributions of Excitatory and Inhibitory Synapses with mGRASP

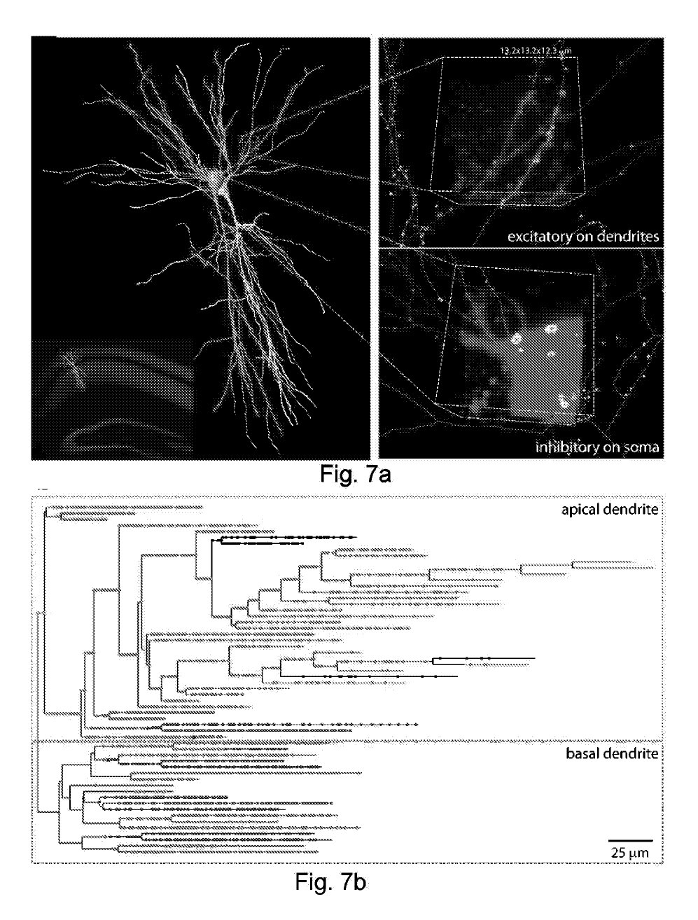

The present automated reconstruction and detection programs can detect synapses and distinguish them from the dendritic compartments of other nearby cells (FIG. 7). An automated method was developed which distinguishes between excitatory and inhibitory synapses based upon the size and shape of mGRASP signals. Taking advantage of reports that all synaptic inputs converging onto the perisomatic area of CA1 pyramidal neurons are inhibitory, fluorescent mGRASP signals were compared from somata and from dendrites of CA1 neurons. mGRASP fluorescent puncta on somata were always large and elliptical, while those on dendrites were small and round (FIG. 7a), supporting our classification system. In parallel, contralateral projections of CA3 interneurons were examined using a genetically manipulated mouse line expressing the Cre recombinase under the control of the endogenous glutamic acid decarboxylase promoter via knock-in (GAD-Cre). Contralateral projections of GAD intereurons were clearly observed in both oriens and radiatum of CA1 when Cre-dependent AAV expressing dTomato was delivered specifically into GAD-interneurons in CA3 (FIG. 6). This indicates large and strong signals from reconstituted mGRASP puncta on the main trunk of the CA1 are inhibitory inputs (FIG. 3a). To investigate the locations and distributions of synapses in depth in dendritic compartments as well as single cells, dendrograms were constructed with separate apical and basal dendrites of CA1 and plotted the locations of synapses on them as detected by mGRASP (FIG. 7b). Detailed descriptions of the synaptic distributions on dendritic compartments will be critical for a full understanding of their contribution to synaptic signaling and dendritic integration.

Discussion

The foregoing studies, experiments and test demonstrate the development, optimization and analysis of mGRASP as a generalizable systelectron microscopic for detecting synapses and specific patterns of connectivity between two regions of the mammalian brain. mGRASP was successfully targeted into synapses without inducing artificial synaptogenesis and proved to detect specifically actual synapses not mere close appositions of axon and dendrite. The lack of artificial synaptogenesis is likely due to the fact that synapse formation and maturation requires not only the interaction of adhesion molecules per se but also the involvement of other signaling factors in a bidirectional trans-synaptic manner. Additionally, a comprehensive framework includes molecular engineering, cellular labeling strategies, and automated reconstruction techniques, that allow optimized mGRASP to facilitate the ease, speed, and accuracy of mapping synaptic locations as well as generating physical maps of neurons that comprise a circuit. The present method and system allow the rapid and precise characterization of synaptic connectivity of neuronal circuits in conditions of health and in neurological disorders that may be caused by abnormal synaptic connectivity (e.g. Autism).

The present method, system and software has advantages over prior techniques, which will now be apparent to those skilled in the art. In recent years, prior optogenetic approaches (i.e. channelrhodopsin) have accelerated the LM-level analysis of synaptic connectivity and synaptic strength, yet these approaches operate at relatively low levels of resolution and can yield ambiguous results. More recent studies have approached “functional connectomics” by combining photon-based calcium imaging with EM-based connectivity mapping in locations such as the mammalian retina and visual cortex. However, only relatively small volumes of brain, especially thin vertical volumes (approximately 50-60 μm) can presently be imaged because EM image acquisition and analysis remains a formidable challenge despite recent innovations.

The present mGRASP system, combined with computer-based 3D reconstruction integrated into a 3D brain atlas, will complement EM and optogenetic approaches, and will greatly accelerate comprehensive studies of long-range synaptic circuits and microcircuits.

The following disclosure provides additional details and the methods and materials used in the preparation of pre- and post-synaptic proteins and other aspects of the present method and system.

Methods

Constructs and Viral Production

The chimeric pre- and post-synaptic mGRASP were designed and synthesized from published sequences (NCBI), using codon optimization for Mus musculus (DNA2.0). Signal peptides and transmembrane domains were predicted by bioinformatics servers (DAS:). Molecular lengths of extracellular domains were simulated using Protein data bank and PyMol. For pre-mGRASP, the signal peptide (1-29 aa) of the nematode β intergrin (PAT-3), the spGFP11, the two flexible linkers (GGGGS), the trimmed human CD4-2 (25-242 aa) including its extracellular and transmembrane domains, and the intracellular domain of the rat neurexin 1β (414-468aa) were sequentially patched. This pre-mGRASP was fused with the optimized mCerulean (pre-mGRASP-mCerulean) for LM study and with the 2A-peptide followed by the optimized dTomato (pre-mGRASP-2A-dTomato) for EM study, respectively. In addition, the pre-mGRASP-Cer-2A-nlsChe was constructed by adding the 2A-peptide and nucleus-localized mCherry to the pre-mGRASP-mCerulean (pre-mGRASP-mCerulean-2A-nlsCherry). For post-mGRASP, the esterase (52-627 amino acids) of mouse neuroligin 1 was truncated and the spGFP1-10 fragment was inserted after its signal peptide (1-49 amino acids). The post-mGRASP was fused with the 2A-peptide followed by the optimized dTomato (post-mGRASP-2A-dTomato) for LM and EM study. These synthesized constructs were cloned into aavCAG vector with the CAG promoter (CMV enhancer, β-actin promoter and regulatory element from the woodchuck hepatitis virus (WPRE)) via BamHI/HindIII digestion. Recombinant adeno-associated viruses (rAAV) were produced and purified by CsCl gradients as described previously. Serotype 1 was used for general infection, while serotype 7 for interneuron infection.

Cre-Dependent ‘on’ and ‘Off’ mGRASP

To make Cre-dependent mGRASP, at first, the faithful flexed AAV vector (aavCAG-Jx) was generated using lox66 and lox71. Two complementary oligos containing lox66, HindIII, EcoRV, BgIII sites and lox71 (JK-lox66/71: 5′gatcATAACTTCGTATAGCATACATTATACGAACGGTAaagcttgatatcagatctATAACTT CGTATAATGTATGCTATACGAACGGTAc3′ (SEQ ID NO:1) and JK-lox66/71: 5′agctgTACCGTTCGTATAGCATACATTATACGAAGTTATagatctgatatcaagcttTACCGT TCGTATAATGTATGCTATACGAAGTTAT3′) (SEQ ID NO:2) were synthesized, annealed, and inserted into aavCAG digested by BamHI and HindIII. For the switch ‘on’ version (aavCAG-Jx-rev-mGRASP), the pre- and post-mGRASP digested by BamHI/HindIII were cloned into the aavCAG-Jx digested by BgIII/HindIII. For the switch ‘off’ version (aavCAG-Jx-mGRASP), the pre- and post-mGRASP digested by Blunted/HindIII were cloned into the pAAV-cag-Jx digested by EcoRV/HindIII. The iCre amplified by PCR³⁰ was cloned into the aavCAG via BamHI/HindIII.

Gene Delivery: In Utero Electroporation and Stereotaxic Virus Injection

All animal procedures were conducted in accordance with protocols approved by the Institutional Animal Care and Use Committee (IACUC) at the Janelia Farm Research Campus, HHMI. DNAs (2 ug/ul) were injected into the right lateral ventricle of embryos from embryonic day 15.5 timed-pregnant C57BL/6J (Charles River). DNAs (aavCAG-iCre or aavCAG-post-mGRASP) were purified using EndoFree Plasmid kit (Quiagen) and dissolved in water. Hippocampal CA1 progenitor cells were transfected via in utero electroporation. Electroporation was achieved with 5 pulses (duration 50 ms, frequency 1 Hz, 43.5 V). Adult mice 2-3 months post-electroporation were deeply anesthetized using an isoflurane-oxygen mixture (1% vol isoflurane/vol O₂) and rAAV was injected via stereotaxic surgery. Stereotaxic coordinates of CA1 were anteroposterior (AP) −2.0 mm relative to bregma, mediolateral (ML) +1.6 mm, and ventral (V) 1.05-1.15 mm and those of CA3 were AP −2.06 mm, ML −2.4 and −2.625 mm, and V 1.95-2.15 mm ventral. 40-50 nl of viral suspension (titer, approximately 2×10¹² pfu/ml measured by QuickTiter AAV Quantitation Kit, Cell Biolabs Inc.) was injected (over 1 min) using a pulled glass micropipette (tip diameter, 10-20 μm; Drummond). To prevent backflow, the micropipette was left in the brain for over 7 min before it was pulled up.

Brain Slice Preparation and Immunostaining for LM and EM

Mice infected with rAAVs were 2-3 weeks later perfused with PBS and 4% paraformaldehyde (PFA) in 0.1 M PB and post-fixed in 4% PFA for 2 h. For mGRASP imaging, brain slices were sectioned to 100-200 μm thickness with a vibratome (VT1200S, Leica) and were then mounted in ProLong Gold antifade reagent (Invitrogen). For fluorescence immunostaining in LM, 50 μm brain slices were blocked in 0.1% Triton X-100 and 10% goat serum in TBS for 1 h at room telectron microscopicperature and incubated with anti-GFP (1:1000, Invitrogen and 1:800, Abcam) in 0.05% Triton X-100 and 5% goat serum in TBS overnight 4° C. followed by Alexa 488-conjugated secondary antibody for 2 h at RT. For immuno-silver-gold staining in EM, 50 μm brain slices were incubated with anti-GFP (1:1000, Invitrogen) in PBS after 10% goat serum blocking overnight 4° C. followed by DAB detection using Vectastain ABC kit according to the manufacturer's protocol (Vector Laboratories). DAB reaction product was silver-gold enhanced by incubating in 2.6% hexamethylenetetramine, 0.2% silver nitrate, and 0.2% sodium borate for 10 min at 60° C. followed by 0.05% gold chloride for 2 min and 3% sodium thiosulfate for 2 min.

Image Acquisition and Data Analysis

Images were acquired with LSM 710, 510 confocal microscopes (Zeiss) equipped with a mortised stage, and a macro zoom system microscope MVX10 (Olympus). The 8-bit tiled images of the hippocampi of brain sections were obtained at 0.4-0.5 μm depth intervals using ×63 1.4 NA, ×40 1.3NA Plan Apochromat oil objectives with 2-4-fold digital zoom controlled by Zeiss software (ZEN 2009) through a motorized stage. To avoid overlapping of signals, three fluorophores (mCerulean, mGRASP, and dTomato) were excited with 405, 488, and 543 nm wavelength and were imaged in emission wavelengths 441-485, 486-554, and 559-639 nm, respectively, using sequential line scanning. Instrument parameter settings were optimized to avoid photobleaching and image saturation. Adjacent stacks had 5-10% overlap to stitch tiled stacks together with precision and trace neurons.

Stitching and Computer-Aided Tracing

The tiles were stitched together to form a single image of the imaging field. Normalized cross correlation (NCC) was used to determine the relative positions of the tiles. Given two tiles, T₁(x,y,z) and T₂(x,y,z), the NCC method calculates the correlation coefficient for every possible 3D displacement A between them. The mathematical formula of the calculation can be written as follows,

$$r ( Δ ) = {\int }_{p ∈ {O}_{Δ}} \frac{[ {T}_{1} ( p ) - μ ( {T}_{1} ( {O}_{Δ} ) ) ] [ {T}_{2} ( p ) - μ ( {T}_{2} ( {O}_{Δ} ) ) ]}{σ ( {T}_{1} ( {O}_{Δ} ) ) σ ( {T}_{2} ( {O}_{Δ} ) )} d p$$

where O_{Δ} is the overlap region between T₁ and T₂ when displaced by the 3D vector with respect to each other, T_{α}(O_{Δ}) is the set of values of the voxels in Lover the region O_{Δ}, and μ(A) and σ(A) are the mean and standard deviations of the values in A. Ideally, the desired relative displacement between T₁ and T₂ is the displacement Δ₀=argmax_{Δ}r(Δ), that maximizes the normalized cross correlation between them. However, for very small overlaps there can be sufficient noise or accidental correlation that they must be eliminated from consideration. Therefore, we simply ignore the NCC values for which O_{Δ} is smaller than 10⁶ voxels, which is a safe threshold because the stacks are imaged to overlap by much more than this.

The present tracing algorithm is based on step-wise cylinder fitting. More details about the tracing algorithm can be found in Zhao et al. 2011. One critical part of the algorithm is to measure how well a cylinder fits to the signal. A cylindrical filter that in cross section is the Laplacian of a Gaussian was used as a neurite in cross section is general a Gaussian diffused spot. We start with U(x,y,z), a “unit cylinder” that has the form:

U(x,y,z)=(1−(x²+y²))e^{-(x}_{²}^{+y}_{²}⁾ where zε[−h/2,h/2]

and then consider the space of all cylindrical filters that can be obtained by taking U and first scaling it symmetrically in x and y by radius r, then rotating it in 3D in any manner desired, and at the last, scaling it along the z axis by a factor α that reflects the anisotropy of the PSF of a typical microscope. In the present, the length of the cylinder, h, is set to 10 pixels.

When a point is selected on the image by a mouse click, the image signal around the point is examined to determine the position of the seed cylinder. Specifically, the initial cylinder is located at the clicked point with its radius r set to 3 pixels and with its axis parallel to the z-axis. In order to move the cylinder closer towards the medial axis of the target branch, it is shifted to the centroid of local signal. After that, the orientation and size of the cylinder are determined by two steps: coarse search and fine tuning. In the first step, coarse search, we discretize the parameter space, checking each sample point to search for the model that gives the highest score when convolved with the signal. In the second step, fine tuning, the retrieved cylinder is further refined by scaling and rotating as above with a gradient descent procedure. Once the first cylinder is set, it is duplicated and the duplicate is advanced along the cylinder's central axis by 5 pixels in the present implementation, where upon it is once again rotated and scaled with a gradient descent procedure to find the best fit to the signal. This walk continues in steps of 5 pixels, as long as the resulting cylinder has a fitting score above a certain threshold.

mGRASP Puncta Detection

The working area is defined by expanding the radius of each reconstructed neurite cylinder with the interval by the length of typical spines (on average 2-2.5 μm). To handle brightness variation in puncta, the detection was conducted sequentially from the gray level of the brightest puncta to the gray level just above background. The final result is the union of the puncta extracted from all the gray levels. For detecting puncta at each gray level, there are three major steps. Note that the parameter values in these steps described below were mainly determined them based on the criterion that both false positive and negative detection rates should be low.

The first step is a rough estimation of puncta locations at a given grey level. The only pixels considered for matching are those (a) greater than the current grey level, and (b) not part of a match to a puncta at a greater grey level. This is accomplished by setting all pixels other than those just described to 0 in a “masked” copy of image volume. Then normalized cross correlation (NCC) between the masked image and a Gaussian kernel is calculated where this kernel is as follows

$$G ( x , y , z ) = \frac{1}{\sqrt{2 π {σ}_{xy} {σ}_{z}}} {e}^{- ( \frac{{x}^{2} + {y}^{2}}{2 {σ}_{xy}^{2}} + \frac{{z}^{2}}{2 {σ}_{z}^{2}} )}$$

where σ_{xy}=0.25 μm and σ_{z}=0.5 μm

This defines a ball with radius 0.25 μm in physical space that matches the minimal size of a punctum. This radius was value was determined by visually checking a population of puncta in our data. Any location whose NCC convolution with the kernel was above the mean and locally maximal was taken as an initial estimate of a puncta location.

The second step refines locations of the puncta and estimates their sizes. This is done through an iterative mean-shift procedure. In this procedure, given a punctum with estimated location p and radius r, a potentially better estimation of the real punctum location is the centroid c of all pixels inside the ball with center p and radius 1.1r. If this gives a better NCC score than the “shift” is accepted and iterated upon, otherwise the current configuration is taken as the final estimate of the punctum.

The final step is to merge puncta estimates that actually cover one large punctum. To determine if two puncta should be merged, the distance between their centers is checked first. If the distance is less than 2 μm, then the intensity profile of any line segment connecting the two centers was examined. The two puncta would be merged if the valley of this line profile was lower than the smallest foreground value. After merging, the center and radius of the new punctum was re-estimated by the same mean-shift procedure, in which the initial center was set to the average of the two original centers and the initial radius was set to the sum of the two original radii.

Reconstructed neurons and detected mGRASP puncta are visualized in V3D.

Although the invention has been described in considerable detail with respect to preferred embodiments, it will be apparent that the invention is capable of numerous modifications and variations, apparent to those skilled in the art, without departing from the spirit and scope of the invention.

### REFERENCES CITED

Throughout this disclosure, numerous references have been cited including references 1-32 listed below. All are herein incorporated by reference.
1. E. Neher and B. Sakmann, Nature 260 (5554), 799 (1976).
2. G. Buzsaki, Nat Neurosci 7 (5), 446 (2004).
3. Davi D Bock, Wei-Chung Allen Lee, Aaron M Kerlin et al., Nature 471 (7337), 177 (2011).
4. C. Sotelo, Nat Rev Neurosci 4 (1), 71 (2003).
5. Yuriy Mishchenko, Tao Hu, Josef Spacek et al., Neuron 67 (6), 1009 (2010).
6. Winfried Denk and Heinz Horstmann, PLoS Biol 2 (11), e329 (2004).
7. G. Knott, H. Marchman, D. Wall et al., J Neurosci 28 (12), 2959 (2008).
8. Kristina D Micheva and Stephen J Smith, Neuron 55 (1), 25 (2007).
9. Jean Livet, Tamily A Weissman, Hyuno Kang et al., Nature 450 (7166), 56 (2007).
10. Ian R Wickersham, David C Lyon, Richard J O Barnard et al., Neuron 53 (5), 639 (2007).
11. Evan H Feinberg, Miri K Vanhoven, Andres Bendesky et al., Neuron 57 (3), 353 (2008).
12. Michael D Gordon and Kristin Scott, Neuron 61 (3), 373 (2009).
13. R Grace Zhai and Hugo J Bellen, Physiology (Bethesda) 19, 262 (2004).
14. Richard Fairless, Henriette Masius, Astrid Rohlmann et al., J Neurosci 28 (48), 12969 (2008).
15. Gamin Dean and Thomas Dresbach, Trends Neurosci 29 (1), 21 (2006).
16. Wannan Tang, Ingrid Ehrlich, Steffen B E Wolff et al., J Neurosci 29 (27), 8621 (2009).
17. Zuwen Zhang and Beat Lutz, Nucleic Acids Res 30 (17), e90 (2002).
18. Ivan Navarro-Quiroga, Ramesh Chittajallu, Vittorio Gallo et al., J Neurosci 27 (19), 5007 (2007).
19. G. Y. Liao and B. Xu, Genesis 46 (6), 289 (2008).
20. T. Zhao, J. Xie, F. Amat et al., Neuroinformatics (2011).
21. J. C. Lacaille, A. L. Mueller, D. D. Kunkel et al., J Neurosci 7 (7), 1979 (1987).
22. T. Klausberger and P. Somogyi, Science 321 (5885), 53 (2008).
23. M Megias, Z Electron microscopicri, T F Freund et al., Neuroscience 102 (3), 527 (2001).
24. T. J. Siddiqui and A. M. Craig, Curr Opin Neurobiol 21 (1), 132.
25. M. K. Belmonte, G. Allen, A. Beckel-Mitchener et al., J Neurosci 24 (42), 9228 (2004).
26. Leopoldo Petreanu, Daniel Huber, Aleksander Sobczyk et al., Nat Neurosci 10 (5), 663 (2007).
27. H. Wang, J. Peca, M. Matsuzaki et al., Proc Natl Acad Sci USA 104 (19), 8143 (2007).
28. Kevin L Briggman, Moritz Helmstaedter, and Winfried Denk, Nature 471 (7337), 183 (2011).
29. Joshua C Grieger, Vivian W Choi, and R Jude Samulski, Nat Protoc 1 (3), 1412 (2006).
30. D. R. Shimshek, J. Kim, M. R. Hubner et al., Genesis 32 (1), 19 (2002).
31. Shoji Komai, Peter H Seeburg, and Pavel Osten, 1 (6), 3166 (2006).
32. R. S. Petralia, Y. X. Wang, F. Hua et al., Neuroscience 167 (1), 68.

What is claimed is:

1. A protein molecule comprising a signal peptide, an extracellular domain, a transmembrane domain, and an intracellular domain, from N-terminus, wherein the signal peptide comprises consecutive 29 to 35 amino acid residues of N-terminus of the nematode β integrin,
the extracellular domain and the transmembrane domain comprises consecutive 218 to 250 amino acids within CD4-2 comprising the amino acids from 25^{th} to 242^{nd} positions, and
the intracellular domain comprises consecutive 55 to 80 amino acids within neurexin 1β comprising the amino acids from 414^{th} to 468^{th} positions.

2. The protein molecule according to claim 1, wherein a linker consisting of 2 to 8 repeats of the amino acid sequence of GGGGS is inserted between the C-terminus of the signal peptide and the N-terminus of the extracellular domain.

3. The protein molecule according to claim 1, consisting essentially of:
the signal peptide comprises an amino acid sequence from 1st to 29th positions of nematode β integrin;
a GGGGSGGGGS linker;
the extracellular domain and the transmembrane domain comprises an amino acid sequence from 25^{th} to 242^{nd} positions of human CD4-2; and
the intracellular domain comprises an amino acid sequence from 414^{th} to 468^{th} positions of rat neurexin 1β.

4. A polynucleotide encoding the protein molecule according to claim 1.

5. An expression vector comprising the polynucleotide according to claim 4.

6. A pharmaceutical composition containing the protein molecule of claim 1, or an expression vector comprising a polynucleotide encoding the protein of claim 1.

7. A method of pre-synaptic delivery of a bioactive material, comprising administering the protein molecule of claim 1 or an expression vector expressing a polypeptide encoding the protein molecule of claim 1, together with a bioactive material, to a patient in need of the administration of the bioactive material.

8. The method according to claim 7, wherein the bioactive material is selected from the group consisting of a gene, an anticancer agent, and an antibiotic.

9. A protein molecule comprising a signal peptide, an extracellular domain, a transmembrane domain, and an intracellular domain, from N-terminus, wherein
the signal peptide comprises consecutive 49 to 60 amino acid residues within neuroligin comprising the amino acids from 1^{st} to 49^{th} positions,
the extracellular domain comprises consecutive 71 to 85 amino acids within neuroligin comprising the amino acids from 627^{th} to 697^{th} positions,
the transmembrane domain comprises consecutive 19 to 30 amino acids within neuroligin comprising the amino acids from 698^{th} to 716^{th} positions, and
the intracellular domain comprises consecutive 127 to 140 amino acids of C-terminus of neuroligin.

10. The protein of claim 9, wherein the neuroligin is mouse neuroligin.

11. The protein molecule according to claim 9, wherein
the signal peptide comprises consecutive 49 amino acid residues from 1^{st} to 49^{th} positions of neuroligin,
the extracellular domain comprises consecutive 71 amino acids from 627^{th} to 697^{th} positions of neuroligin,
the transmembrane domain comprises consecutive 19 amino acids from 698^{th} to 716^{th} positions of neuroligin, and
the intracellular domain comprises consecutive 127 amino acids of C-terminus of neuroligin.

12. A polynucleotide encoding the protein molecule according to claim 9.

13. An expression vector comprising the polynucleotide according to claim 12.

14. A pharmaceutical composition containing the protein molecule of claim 9 or a polynucleotide encoding the protein molecule of claim 9.

15. A method of post-synaptic delivery of a bioactive material, comprising administering the protein molecule of claim 9 or a polynucleotide encoding the protein of claim 9, together with a bioactive material, to a patient in need of the administration of the bioactive material.

16. The composition according to claim 15, wherein the bioactive material is selected from the group consisting of a gene, an anticancer agent, and an antibiotic.

17. A method of preventing or treating a cranial nerve disorder, comprising administering at least one of the protein molecules of claim 1 or an expression vector expressing a polynucleotide encoding the protein of claim 1, and a gene or drug for preventing or treating the cranial nerve disorder, to a patient in need of preventing or treating a cranial nerve disorder.

18. A method of preventing or treating a cranial nerve disorder, comprising administering at least one of the protein molecules of claim 9 or an expression vector expressing a polynucleotide encoding the protein of claim 9, and a gene or drug for preventing or treating the cranial nerve disorder, to a patient in need of preventing or treating a cranial nerve disorder.

19. The method according to claim 17, further comprising the step of identifying the patient in need of preventing or treating a cranial nerve disorder, prior to the administering step.

20. The method according to claim 18, further comprising the step of identifying the patient in need of preventing or treating a cranial nerve disorder, prior to the administering step.

21. The method according to claim 17, wherein the cranial nerve disorder is selected from the group consisting of Alzheimer's disease, autism spectrum disorder, Parkinson's disease, addiction, depression, amyotrophic lateral sclerosis (ALS), and attention deficit hyperactivity disorder.

22. The method according to claim 18, wherein the cranial nerve disorder is selected from the group consisting of Alzheimer's disease, autism spectrum disorder, Parkinson's disease, addiction, depression, amyotrophic lateral sclerosis (ALS), and attention deficit hyperactivity disorder.

23. A method for visualization of neuron, comprising administering the protein molecule of claims 1, or an expression vector expressing a polynucleotide encoding the protein of claim 1, to a subject, wherein the protein is labeled with a fluorescent material, or the expression vector comprises a gene for a fluorescent protein at the C-terminus of a signal peptide.

24. A method for visualization of neuron, comprising administering the protein molecule of claim 9, or an expression vector expressing a polynucleotide encoding the protein of claim 9, to a subject, wherein the protein is labeled with a fluorescent material, or the expression vector comprises a gene for a fluorescent protein at the C-terminus of a signal peptide.

25. The method according to claim 23, wherein the method is for pre-synaptic visualization, and comprises administering the protein molecule of claim 1, or an expression vector expressing a polynucleotide encoding the protein molecule of claim 1, to a subject, wherein the protein is labeled with a fluorescent material, or the expression vector comprises a gene for a fluorescent protein between a signal peptide and an extracellular domain or a linker.

26. The method according to claim 24, wherein the method is for post-synaptic visualization, and comprises administering the protein molecule of claim 9, or an expression vector expressing a polynucleotide encoding the protein of claim 9, to a subject, wherein the protein is labeled with a fluorescent material, or the expression vector comprises a gene for a fluorescent protein between a signal peptide and an extracellular domain.

27. The method according to claims 23, wherein the subject is a cell, a tissue, or an organ, which is isolated or not isolated from a living body of an animal.

28. The method according to claims 24, wherein the subject is a cell, a tissue, or an organ, which is isolated or not isolated from a living body of an animal.

29. A pharmaceutical composition for treating, minimizing or avoiding a cranial nerve disorder, comprising the protein molecule of claim 1, or an expression vector expressing a polynucleotide encoding the protein of claim 1, and a gene or drug for treating, minimizing or avoiding the cranial nerve disorder.

30. A pharmaceutical composition for treating, minimizing or avoiding a cranial nerve disorder, comprising the protein molecule of claim 9, or an expression vector expressing a polynucleotide encoding the protein of claim 9, and a gene or drug for treating, minimizing or avoiding the cranial nerve disorder.

31. A composition for visualization of neuron, comprising the protein molecule of claim 1, or an expression vector expressing a polynucleotide encoding the protein of claim 1, wherein the protein is labeled with a fluorescent material, or the expression vector comprises a gene for a fluorescent protein at the C-terminus of a signal peptide.

32. A composition for visualization of neuron, comprising the protein molecule of claim 9, or an expression vector expressing a polynucleotide encoding the protein of claim 9, wherein the protein is labeled with a fluorescent material, or the expression vector comprises a gene for a fluorescent protein at the C-terminus of a signal peptide.

33. The composition according to claim 31, wherein the composition is for pre-synaptic visualization, and contains the protein molecule of claim 1, an expression vector expressing a polypeptide encoding the protein of claim 1, wherein the protein is labeled with a fluorescent material, or the expression vector comprises a gene for a fluorescent protein between a signal peptide and an extracellular domain or a linker.

34. The composition according to claim 32, wherein the composition is for post-synaptic visualization, and contains the protein molecule of claim 9, or an expression vector expressing a polynucleotide encoding the protein of claim 9, wherein the protein is labeled with a fluorescent material, or the expression vector comprises a gene for a fluorescent protein between a signal peptide and an extracellular domain.

34. A protein molecule of claim 1, or an expression vector expressing a polynucleotide encoding the protein of claim 1 for the use of visualization of neuron, wherein the protein is labeled with a fluorescent material, or the expression vector comprises a gene for a fluorescent protein at the C-terminus of the signal peptide.

35. A protein molecule of claims 1, or an expression vector expressing a polypeptide encoding the protein of claim 1 for the use of pre-synaptic visualization, wherein the protein is labeled with a fluorescent material, or the expression vector comprises a gene for a fluorescent protein between a signal peptide and an extracellular domain or a linker.

36. A protein molecule of claim 9, or an expression vector expressing a polynucleotide encoding the protein of claim 9 for the use of visualization of neuron, wherein the protein is labeled with a fluorescent material, or the expression vector comprises a gene for a fluorescent protein at the C-terminus of the signal peptide.

37. A protein molecule of claims 9, or an expression vector expressing a polypeptide encoding the protein of claim 9 for the use of post-synaptic visualization, wherein the protein is labeled with a fluorescent material, or the expression vector comprises a gene for a fluorescent protein between a signal peptide and an extracellular domain.

38. A method for three dimensional reconstructing neuron morphologies, said method comprising:
(a) selecting a starting seed point by a user using an computer processor;
(b) placing a first cylinder on the seed via the computer processor;
(c) adjusting orientation(s) and shape of the cylinder by the computer processor by maximizing a correlation between the cylinder and a local intensity distribution of an image of neurons, to thereby setting the first cylinder;
(d) duplicating the first cylinder and advancing the duplicated cylinder along a central axis of the duplicated cylinder, by the processor;
(e) adjusting the shape and orientation of each cylinder using a gradient descent algorithm to optimize the a match to an underlying neurite in the image;
(f) adding additional cylinders, one by one along their central axis, in both directions, until the image of the neurite is completely covered; and
(g) applying minimal spanning tree (MST) to connect adjacent neurites and to exclude non-tree structures like loops.


SEQUENCE LISTING (2 sequences)

SEQ ID NO: 1 - DNA, 91 bp, Artificial Sequence
  synthesized oligo
gatcataacttcgtatagcatacattatacgaacggtaaagcttgatatcagatctataacttcgtataatgtatgctatacgaacggtac

SEQ ID NO: 2 - DNA, 91 bp, Artificial Sequence
  synthesized oligo
agctgtaccgttcgtatagcatacattatacgaagttatagatctgatatcaagctttaccgttcgtataatgtatgctatacgaagttat